(19) United States
(12) Patent Application Publication
(10) Pub. No.: US 2026/0297010 A1
(43) Pub. Date: Oct. 1, 2026

(54) PROCESS FOR THE PRODUCTION OF CABOXYLIC ACIDS AND CARBONYL COMPOUNDS BY UTILIZING CARBON DIOXIDE-RICH FEEDSTOCK

(71) Applicant: KT TECH S.P.A., Roma (IT)

(72) Inventors: Luca Eugenio Riccardo Basini, Milano (IT); Antonio Batistini, Camaiore (IT); Michele Colozzi, Roma (IT); Emma Palo, Battipaglia (IT)

(21) Appl. No.: 19/489,046

(22) PCT Filed: May 30, 2023
(86) PCT No.: PCT/EP2023/064425

§ 371 Date: Dec. 1, 2025

(51) Int. Cl. C07C 51/12; B01J 8/02; C01B 3/386; C01B 3/56; C07C 29/151; C07C 68/00

(52) U.S. Cl. CPC C07C 51/12; B01J 8/0278; B01J 8/0285; C01B 3/386; C01B 3/56; C07C 29/151; C07C 68/00; B01J 2208/00176; C01B 2203/0261; C01B 2203/042; C01B 2203/047; C01B 2203/061; C01B 2203/0883; C01B 2203/1017; C01B 2203/1241

(57) ABSTRACT

Process to produce carboxylic acids and carbonyl compounds comprising the steps of: a) producing a syngas by means of a short contact time catalytic partial oxidation process which uses a feedstock comprising a mixture of carbon dioxide included in natural gas and other gases, and/or carbon dioxide captured from gaseous streams, said carbon dioxide being from 5 to 50% v/v of said mixture; and air, oxygen, air enriched with oxygen, and optionally steam; b) using said syngas for the production of a compound selected from the group consisting of carboxylic acids, alcohols, carbonyl compounds and carbonates. Also described is an apparatus to carry out the process.

The present invention relates to a process for the production of carboxylic acids, carbonates, carbonyl compounds and alcohol species and other products obtained with process solutions including the production and use of synthesis gas having low hydrogen vs. carbon monoxide ratios obtained by utilizing carbon dioxide-rich feedstock. The invention also relates to an apparatus for carrying out this process.

### BACKGROUND OF THE INVENTION

The CO₂ concentration in the atmosphere has overcome, in 2016, the 400 ppm after having remained between 100 ppm and 300 ppm for thousands of years before, during and after the industrial revolution periods.

The reduction of greenhouse gases (GHG) concentration, whose main components are CO₂ and methane, is crucial and requires a systemic approach for an efficient use of the primary energy sources in which the percentage of renewable energy needs to be increased while the share of fossil sources need to be reduced.

From another perspective, CO₂ is also a synthon but being a highly stable molecule, its transformations require a relevant energy input and/or complex biological cycles.

The present invention provides innovative solutions for utilizing CO₂ in the synthesis gas production and subsequently for utilizing the produced syngas for chemicals and fuels production.

### PRIOR ART

Currently, the synthesis gas (syngas) with low H₂ vs CO ratios is obtained with steam-CO₂ reforming technologies utilizing large amounts of CO₂ and often referred to as dry reforming technologies. These technologies are limited in their operation windows by carbon formation reactions.

As mentioned above, in steam-CO₂ reforming, particularly in high pressure conditions, the occurrence of carbon formation reactions onto the catalyst surfaces cause limitations in the operation conditions; these limitations particularly affect the possibility of lowering or avoiding the steam content in the reactant mixtures while operation in low steam vs carbon (moles of carbon in the hydrocarbon feedstock) would be particularly advantageous for producing synthesis gas with low H₂ vs CO ratios.

In other words, the production processes utilizing steam-CO₂ reforming and particularly dry reforming are limited by thermodynamic and kinetic affinity towards carbon formation reactions occurring at the surfaces of the catalytic species.

Steam-CO₂ reforming and dry reforming [1] are also examined as a possible solution for defining carbon capture and utilization processes as well as other processes which utilize CO₂ as synthon, such as: i) the urea synthesis [2] particularly from blue NH₃, ii) the methanol synthesis from direct hydrogenation of CO₂ [3] and iii) the CO₂ methanation to synthetic natural gas (SNG) [4] and liquid hydrocarbons [5].

$$\begin{matrix}{CH}_{4} + {CO}_{2} = 2 CO + 2 {H}_{2} & Δ H ° = 247.3 kJ / mol\end{matrix} \qquad [ 1 ]$$

$$\begin{matrix}2 {NH}_{3} + {CO}_{2} = {NH}_{2} {CONH}_{2} + {H}_{2} O & Δ H ° = - 133.5 kJ / mol\end{matrix} \qquad [ 2 ]$$

$$\begin{matrix}3 {H}_{2} + {CO}_{2} = C {H}_{3} OH + {H}_{2} O & Δ H ° = - 48 kJ / mol\end{matrix} \qquad [ 3 ]$$

$$\begin{matrix}{CO}_{2} + 4 {H}_{2} = {CH}_{4} + 2 {H}_{2} O & Δ H ° = - 164 kJ / mol\end{matrix} \qquad [ 4 ]$$

$$n {CO}_{2} + ( 4 n + 2 ) {H}_{2} = {C}_{n} {H}_{2 n + 2} + 2 n {H}_{2} O \qquad [ 5 ]$$

However, except for the urea production [2], the other pathways still appear as rather inefficient. Indeed, direct hydrogenation of CO₂ into methanol [3], into synthetic natural gas [4], and synthetic liquid hydrocarbons [5] still show mayor thermodynamic limitations and relatively low selectivity features.

Concerning the production of CO-rich syngas by steam-CO₂ reforming and/or dry reforming, it is reported that this process is affected by the possible deactivation of the catalysts due to the carbon formation reactions [6-9] particularly at the industrially relevant pressures between 15-40 bar.

$$\begin{matrix}2 CO = {CO}_{2} + C & Δ H ° = - 171 kJ / mol\end{matrix} \qquad [ 6 ]$$

$$\begin{matrix}CO + {H}_{2} = {H}_{2} O + C & Δ H ° = 131 kJ / mol\end{matrix} \qquad [ 7 ]$$

$$\begin{matrix}{CH}_{4} = 2 {H}_{2} + C & Δ H ° = 75 kJ / mol\end{matrix} \qquad [ 8 ]$$

$${C}_{n} {H}_{m} ( g ) = olefins ( g ) = coke ( s ) \qquad [ 9 ]$$

Reactions [6-9] are the primary sources of carbon that can be divided into three typologies: i) whisker carbon, ii) gum (encapsulating carbon), and iii) pyrolytic carbon.

Gum formation and pyrolytic carbon are especially associated with the presence of hydrocarbons with more than two carbon atoms in the feedstock and can be avoided by removing these molecules with a pre-reformer unit upstream dry reforming.

Instead, the production of whisker carbon can only be avoided by operating in condition having a low thermodynamic affinity towards this species that once formed bring to the catalyst pellet destruction and to the plant shut down due to the necessity of avoiding that the heat flow from the furnace not adsorbed by the endothermic reactions leads to tubes overheating and fracturing.

Therefore, one of the primary tasks in the development of a steam-CO₂ reforming and/or dry reforming processes, is the careful theoretical and experimental analysis of the allowed carbon free operating conditions.

Significant research has been conducted for limiting, with appropriate catalysts definition, the carbon formation reactions. However, whisker carbon formation remains primarily dictated by thermodynamics instead of kinetics and its avoidance can be obtained only in limited operation conditions.

Nickel, cobalt, and noble metal catalysts, among others, have been extensively studied as potential catalysts for steam-CO₂ reforming and dry reforming, with nickel being the most investigated system, as this is, cost-wise, the most attractive catalyst.

Noble metal catalysts are also utilized since they typically have a lower kinetic affinity for carbon formation and a higher activity for steam and CO₂ reforming reactions.

In addition, it is noted that the CO₂ consumption features in steam-CO₂ and dry reforming is limited by the emissions determined by the heating furnaces. Indeed, the CO₂ reforming reaction [1] requires ca. 22% of enthalpy more than steam reforming [10] and this heat is currently provided in heating furnaces with reactions such as [11]

$$\begin{matrix}{CH}_{4} + {H}_{2} O = 3 {H}_{2} + CO & Δ H ° = + 206 kJ /\end{matrix} moles \qquad [ 10 ]$$

$$\begin{matrix}{CH}_{4} + 2 {O}_{2} + 4 {N}_{2} = {CO}_{2} + 2 {H}_{2} O + 4 {N}_{2} & Δ H ° = - 802 kJ /\end{matrix} mole \qquad [ 11 ]$$

Since the heat transferred into the reaction inside the reforming tubes is roughly the 50% of the heat released by the combustion reactions [11], it can be estimated that per any CO₂ molecule consumed with reaction [1] ca 0.6 CO₂ molecules would be produced when the reaction heat is provided by burning CH₄ molecules.

Despite these difficulties, steam-CO₂ reforming processes in which some steam addition prevents the above-mentioned carbon formation reactions have been defined and commercialized, while pure dry reforming is not currently commercialized.

In conclusion, it is mentioned that TOPSOE has developed a process solution in which a hot CO₂ containing stream is added to the synthesis gas produced by a steam reformer operating at low steam vs. carbon ratios. In this way a CO₂ enriched synthesis gas is transformed in a subsequent Adiabatic POst Converter (APOC) reactor in conditions in which the thermodynamic affinity towards the carbon formation reactions is greatly reduced. Indeed, in the APOC reactor the CO₂ is transformed with a reverse water gas shift reaction into CO and H₂O. The use of the APOC provides then a method for tailoring a synthesis gas composition in a wide range of H₂ vs CO ratio minimizing the risk of carbon formation.

In view of the foregoing, there is the need of innovative process and technology solutions that overcome the steam-CO₂ reforming and the dry reforming drawbacks related to the occurrence of carbon formation reactions.

### SUMMARY OF THE INVENTION

An aspect of the present invention is a process to produce carboxylic acids and carbonyl compounds, alcohols and other compounds through the production and use of a low hydrogen vs carbon monoxide synthesis gas comprising the steps of:
a) producing a syngas by means of a short contact time catalytic partial oxidation process which uses a feedstock comprising an organic component and an inorganic component, wherein:
   i) said organic component is a mixture comprising carbon dioxide included in one or more of natural gas, associated gases, off-gases of industrial chemistry and/or refining activities, blast furnace gases, coke oven gases and direct reduction gases produced in the reduction of iron ores, biogas and carbon dioxide captured from gaseous streams, said carbon dioxide being from 5 to 50% v/v of said mixture;
   ii) said inorganic component comprises one or more of air, oxygen, and air enriched with oxygen, and optionally steam;
   iii) said oxygen is obtained from electrolysis of water or from air; and
   iv) said short contact time catalytic partial oxidation process is carried out in a reactor on a catalytic bed comprising a portion with truncated cone geometry operated at a gas hourly space velocity from 15,000 to 500,000 h⁻¹;
   v) said syngas has a hydrogen/CO ratio from 0.5 to 2;
b) using said syngas for the production of a compound selected from the group consisting of carboxylic acids, alcohols, carbonyl compounds and carbonates.

Another aspect of the invention is an apparatus for producing synthetic hydrocarbon compounds with the process defined above, comprising a short contact time catalytic partial oxidation reactor including a first portion having a cylindrical shape consisting of an inlet a mixing zone and a thermal shield zone, a second portion containing a catalytic bed having a truncated cone shape, and a third portion having a cylindrical shape with a diameter greater than the diameter of said first cylindrical portion, followed by a second thermal shield zone, wherein:
a) in said second portion having the shape of a truncated cone the upper base is smaller than the bottom base;
b) said upper base of said truncated cone is joined to said first cylindrical portion and said bottom base is joined to said third cylindrical portion; and
c) the external angle (α) of said truncated cone at the upper base is lower than 85° and preferably lower than 75°.

The term “short contact time catalytic partial oxidation” (SCT-CPO) has a well-defined meaning either in the scientific, technical, and patent literature. As used in the present invention, however, certain aspects are quite specific for what concerns the combination of the characteristics of the reactor and the operation conditions.

### DETAILED DESCRIPTION OF THE INVENTION

The apparatus according to the present invention overcome the drawbacks of the prior art by utilizing short contact time-catalytic partial oxidation (SCT-CPO) reactors with a catalytic bed having a truncated cone geometry placed between thermal shields.

This reactor configuration, an appropriate choice of the catalyst composition and shape and an appropriate selection of operation conditions does not determine the occurrence of the carbon formation reactions at the catalyst surfaces also in the presence of large amounts of CO₂ in the reactant mixture.

Indeed, it has also been found that with the utilization of the SCT-CPO reactor solutions here described, the presence of the CO₂ inside the reactant molecules mixture, does not determine the occurrence of carbon formation reactions onto the catalysts surfaces but also inhibits the occurrence of radical reactions in the gas phase that, particularly in high pressure conditions, lead to unsaturated hydrocarbon formation that can further aggregate and decompose producing carbonaceous deposits.

Hence in the SCT-CPO systems, here described the carbon formation reactions observed in steam-CO₂ reforming and dry reforming cannot be originated.

It is stressed the point that only with the adopted and here described reactor solutions, we have found that: i) the presence of CO₂ in the reactant mixture inhibits the propagation of the unselective radical reactions inside the reactant/product gaseous mixture progressing inside the SCT-CPO reactor while ii) the CO₂ participates, mainly through the reverse water gas shift reaction (RWGS), the heterogeneous chemistry inside the catalytic bed after that relevant amounts of hydrocarbons have been already transformed into synthesis gas.

Other CPO reactor solutions utilizing tubular and fixed bed reactors, fluidized bed, moving beds, or bubbling bed reactors cannot be applied in the conditions here described.

The SCT-CPO technology is described in numerous patent documents, including WO2016016257 (A1), WO2016016256 (A1), WO2016016253 (A1), WO2016016251 (A1), WO 2011151082, WO 2009065559, WO 2011072877, US 2009127512, WO 2007045457, WO 2006034868, US 2005211604, WO 2005023710, WO 9737929, EP 0725038, EP 0640559.

Extensive technical and scientific literature has been produced on SCT-CPO and mentioned, for example in:
¹“Issues in H2 and synthesis gas technologies for refinery, GTL and small and distributed industrial needs”; Basini, Luca, Catalysis Today, 106 (1-4), p. 34, October 2005

“Fuel rich catalytic combustion: Principles and technological developments in short contact time (SCT) catalytic processes”; Basini, L.; Catalysis Today, 117 (4), 384-393; DOI: 10.1016/j.cattod.2006.06.043 Published: 15 Oct. 2006

“Natural Gas Catalytic Partial Oxidation: A Way to Syngas and Bulk Chemicals Production|IntechOpen”; G. Iaquaniello, E. Antonetti, B. Cucchiella, E. Palo, A. Salladini, A. Guarinoni, A. Lainati and L. Basini; http://dx.doi.org/10.5772/48708

“Short Contact Time Catalytic Partial Oxidation (SCT-CPO) for Synthesis Gas Processes and Olefins Production”; L. E. Basini, A. Guarinoni, Ind. Eng. Chem. Res. 2013, 52, 17023-17037; https://doi.org/10.1021/ie402463m.

We have found that the utilization of specific SCT-CPO reactors and operation conditions improves the possibility of transforming CO₂ rich stream into a CO rich syngas for producing chemical and fuels completely avoiding the occurrence of conditions in which the carbon formation reactions are produced either for kinetics or thermodynamic reasons.

Moreover, we have found that, inside the SCT-CPO reactor according to the invention, the CO₂ is transformed mainly through the RWGS reaction after that the gaseous hydrocarbons and the oxygen containing flows have been largely converted into a mixture of H₂ and CO. In this way within the same SCT-CPO reactor, it is possible to produce the same result that would be obtained by combining the steam-CO₂ reforming or dry reforming and APOC reactors.

It is also mentioned that this technology requires moderate pre-heating of the reactant mixtures, and this pre-heating can be achieved by recovering the heat obtained by cooling the produced synthesis gas avoiding the utilization of fire heaters and hence avoiding the CO₂ emissions that this use would produce.

The use of the specific solutions for SCT-CPO reactors and operation conditions provides a unique mean for obtaining CO rich syngas production suitable for chemical and/or other products by integrating substantial amounts of CO₂ into the final products.

More specifically we have found that the SCT-CPO technology solutions here described allow the utilization of hydrocarbon feedstock also including relevant amounts of CO₂ in a wide range of pressure conditions (1-80 ATM and preferentially between 5-35 ATM) without incurring in the carbon formation reactions. Moreover, we have found that the here described solutions allow the operation of SCT-CPO reactors with a moderate pre-heating of the reactants that can be provided with gas-gas heat exchangers recovering the heat from the produced synthesis gas.

In this way the syngas can be produced, by utilizing the CO₂ as a synthon, with a very low H₂ vs CO ratio suitable for chemical processes such as, the synthesis of the acetic acid, the hydroformylation of olefins, the production of other chemicals such as the Methylene Diphenyl Diisocyanate (MDI), Methyl Methacrylate (MMA), Dimethyl Carbonate (DMC) Dimethyl Ether (DME).

The invention is described also with reference to the accompanying drawings, wherein:

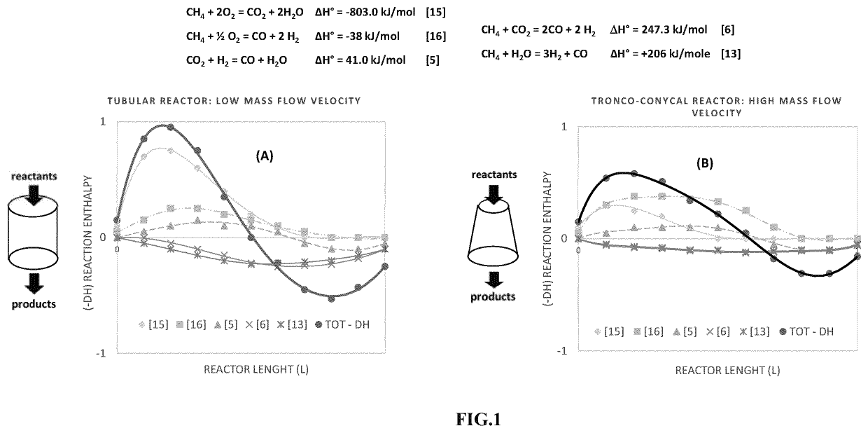

FIG. 1. (A) Qualitative drawing of the enthalpy/temperature contributions determined by reactions [1], [10] and [12-14] occurring along a tubular reactor including a fixed catalytic bed operated at low mass flow velocity; the solid line represents the total enthalpy variation.
(B) Qualitative drawing of the enthalpy/temperature contributions determined by reactions [1], [10] and [12-14] occurring along a truncated cone geometry including a fixed catalytic bed operated at high mass flow velocity; the solid line represents the total enthalpy variation

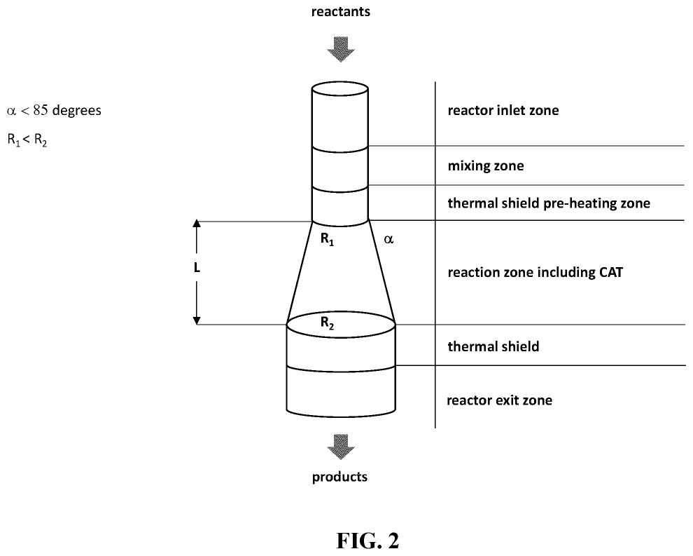

FIG. 2. Scheme of the internal zones of a SCT-CPO reactor with a truncated cone reaction zone with elements on the geometry of the truncated cone zone

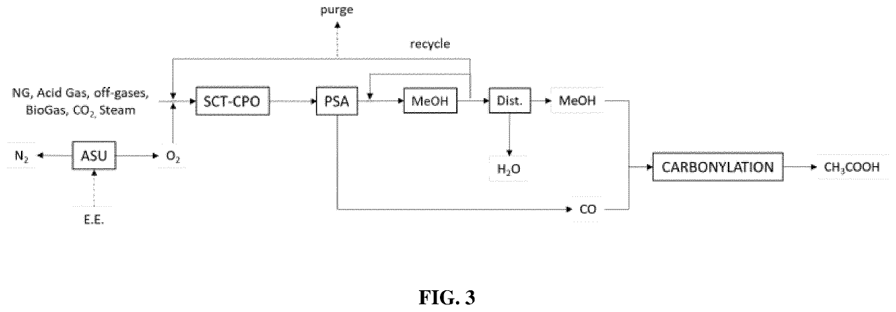

FIG. 3. Describes a process scheme for integrating CO₂ rich streams into the production, via SCT-CPO, of a syngas with low H₂ vs CO ratios (H₂/CO ca 1 v/v) useful for the acetic acid synthesis through methanol carbonylation. In this case some CO is separated with a dedicated pressure swing adsorption (PSA) unit for obtaining a synthesis gas stream with a methanol module ca. 2 v/v, useful for methanol production while, the separated CO is utilized for methanol carbonylation

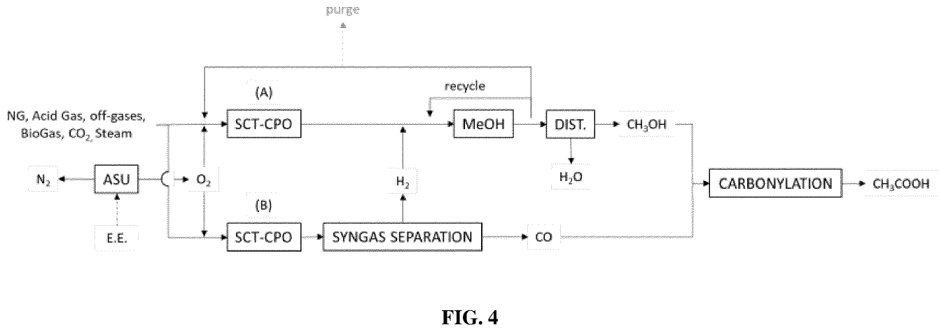

FIG. 4. Describes a process scheme for integrating CO₂ rich streams into acetic acid through the intermediate production, via SCT-CPO, of a syngas with low H₂ vs CO ratios (H₂/CO ca 1 v/v). The scheme includes two SCT-CPO reactors (A) and (B) operating in parallel. The components of the syngas produced by reactor (B) are separated and the H₂ containing stream is added to the syngas produced by reactor (A) for achieving a composition suitable for the methanol synthesis, while the CO is used for the following methanol carbonylation step

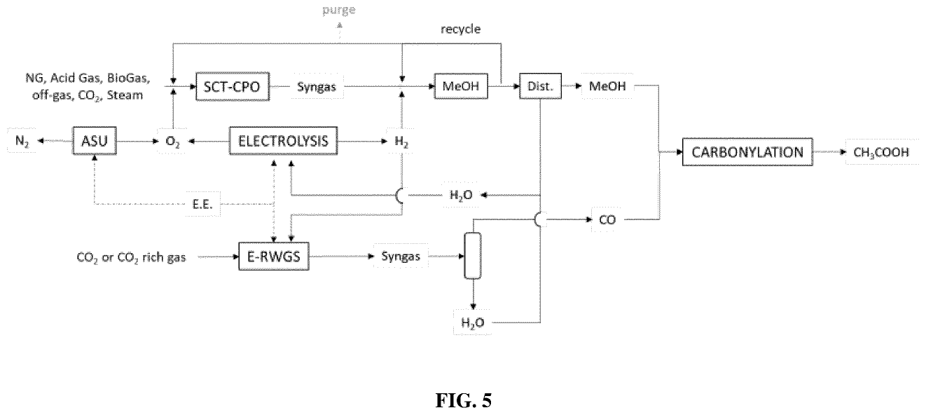

FIG. 5. Describes a process scheme for integrating CO₂ rich streams into the production, via SCT-CPO, of a syngas with low H₂ vs CO ratios (H₂/CO ca 1 v/v) useful for the acetic acid synthesis through methanol carbonylation. In this case some H₂ provided by steam electrolysis can be added to the synthesis gas for achieving a methanol module ca. 2 v/v useful for the methanol synthesis, while the CO useful for methanol carbonylation is obtained from CO₂ and H₂ by utilizing an electrified reverse water gas shift (E-RWGS) reactor

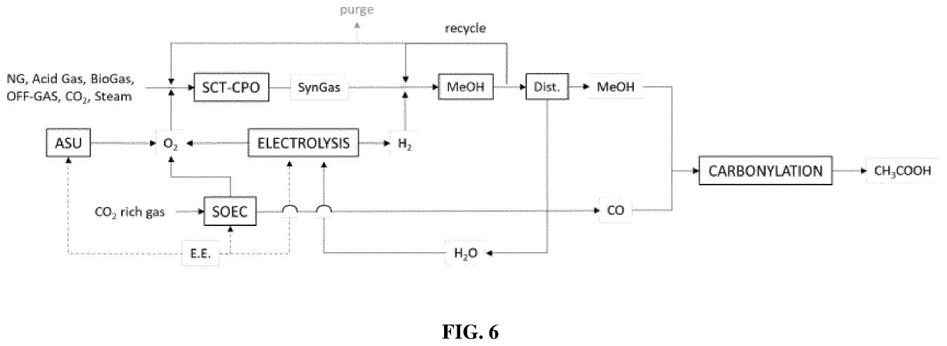

FIG. 6. Describes a process scheme for integrating CO₂ rich streams into the production, via SCT-CPO, of a syngas with low H₂ vs CO ratios (H₂/CO ca 1 v/v) useful for the acetic acid synthesis through methanol carbonylation. In this case some H₂ provided by steam electrolysis is added to the synthesis gas for achieving a methanol module ca. 2 v/v useful for the methanol synthesis, while the CO useful for methanol carbonylation is obtained from CO₂ electrolysis performed with a solid oxide electrolysis cell (SOEC)

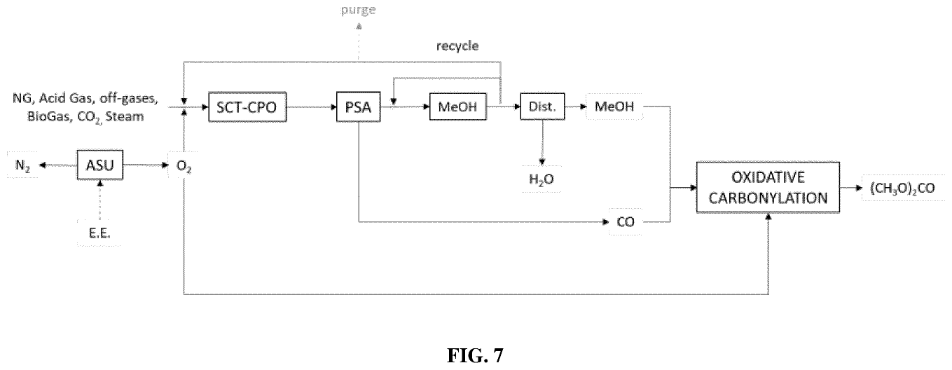

FIG. 7. Describes a process scheme for integrating CO₂ rich streams into the production, via SCT-CPO, of a syngas with low H₂ vs CO ratios (H₂/CO ca 1 v/v) useful for the DMC synthesis through the oxidative methanol carbonylation. In this case some CO is separated with a pressure swing adsorption unit for obtaining a synthesis gas stream with a methanol module ca. 2 v/v, while the separated CO is utilized for methanol carbonylation

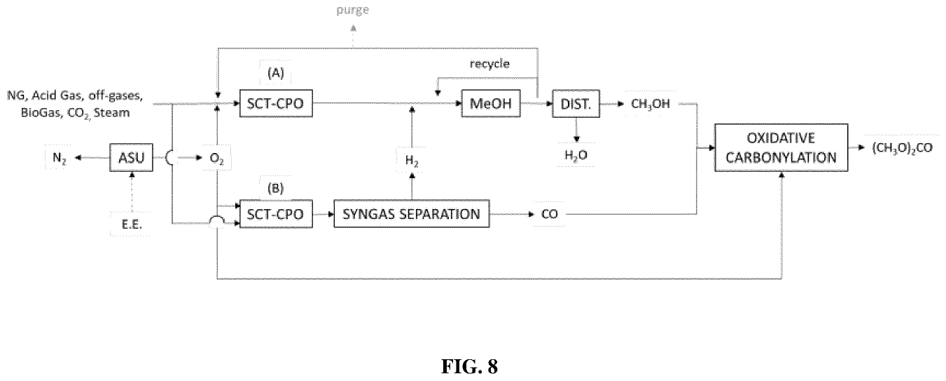

FIG. 8. Describes a process scheme for integrating CO₂ rich streams into DMC through the intermediate production, via SCT-CPO, of a syngas with low H₂ vs CO ratios (H₂/CO ca 1 v/v). The scheme includes two SCT-CPO reactors (A) and (B) operating in parallel. The components of the syngas produced by reactor (B) are separated and the H₂ containing stream is added to the syngas produced by reactor (A) for achieving a composition suitable for the methanol synthesis, while the CO is used for the following oxidative carbonylation step

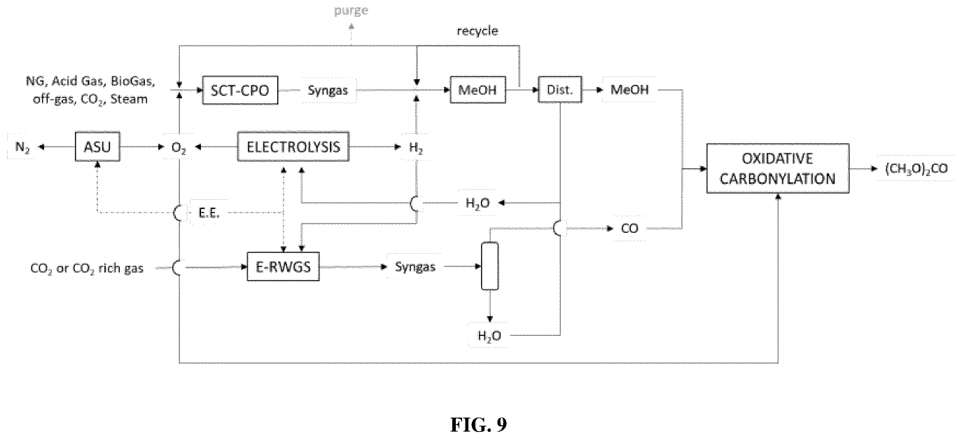

FIG. 9. Describes a process scheme for integrating CO₂ rich streams into the production, via SCT-CPO, of a syngas with low H₂ vs CO ratios (H₂/CO ca 1 v/v) useful for DMC production through the oxidative carbonylation of methanol. In this case some H₂ provided by steam electrolysis is added to the synthesis gas for achieving a methanol module ca. 2 v/v useful for the methanol synthesis, while the CO useful for methanol carbonylation is achieved from CO₂ and H₂ by utilizing an electrified reverse water gas shift (E-RWGS) reactor

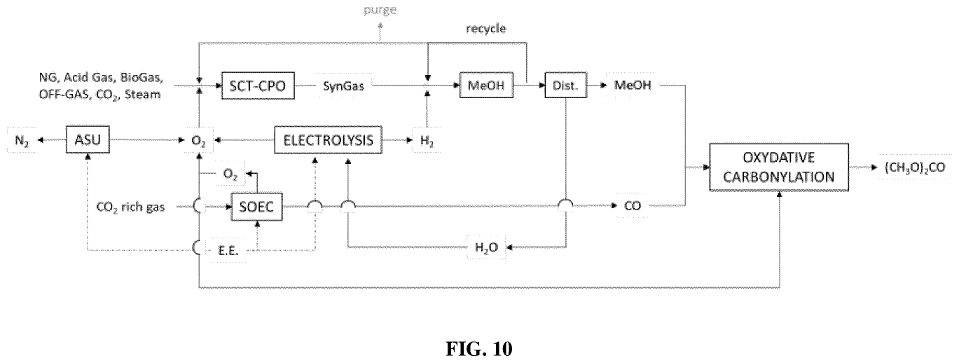

FIG. 10. Describes a process scheme for integrating CO₂ rich streams into the production, via SCT-CPO, of a syngas with low H₂ vs CO ratios (H₂/CO ca 1 v/v) useful for the DMC synthesis through methanol oxidative carbonylation. In this case some H₂ provided by steam electrolysis is added to the synthesis gas for achieving a methanol module ca. 2 v/v useful for the methanol synthesis, while the CO useful for methanol carbonylation is achieved from CO₂ electrolysis inside an SOEC

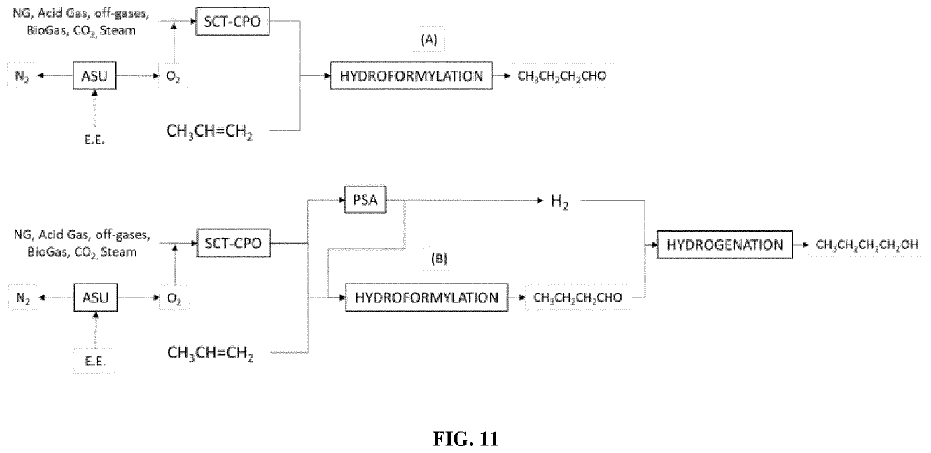

FIG. 11. Describes a process scheme for integrating CO₂ rich streams into the production, via SCT-CPO, of a syngas with low H₂ vs CO ratios useful for the hydroformylation of alkenes (in this case propene) for producing (A) the corresponding aldehyde (in this case n-butyraldehyde) and (B) the corresponding oxo-alcohol (in this case n-butanol)

The thermo-chemical properties of the reaction environment produced in short contact time conditions during the SCT-CPO in a heterogeneous catalytic fixed bed reactor fed with a premixed CH₄, Steam, CO₂ and O₂ stream, can be discussed considering the system composed by the by equations [1], [8], [10-12]

$${CH}_{4} + 2 {O}_{2} = {CO}_{2} + 2 H 2 O Δ H ° = - 803. kJ / mol \qquad [ 12 ]$$

$${CH}_{4} + 1 / 2 {O}_{2} = CO + 2 {H}_{2} Δ H ° = - 38 kJ / mol \qquad [ 13 ]$$

$$CO + {H}_{2} O = {CO}_{2} + {H}_{2} Δ H ° = - 41. kJ / mol \qquad [ 14 ]$$

$${CH}_{4} + C {O}_{2} = 2 CO + 2 {H}_{2} Δ H ° = 247.3 kJ / mol \qquad [ 1 ]$$

$${CH}_{4} + {H}_{2} O = 3 {H}_{2} + CO Δ H ° = + 206 kJ / mole \qquad [ 10 ]$$

Clearly the exothermic total oxidation reaction [12] would have highest probability to be localized at the beginning of the bed, while the endothermic steam-CO₂ reforming [1] and [10] and the mildly exothermic (WGS) reactions [14] would have the highest probability of occurring in following zone. Noteworthy by increasing the reaction temperature above 830° C. reaction [14] is shifted on the left side and a slightly endothermic reverse water gas shift (RWGS) reaction is favoured together with steam-CO₂ reforming [1] and [10].

Moreover, we found that the extent and the localization of these reactions was greatly affected by physical and chemical factors. Total combustion [12] has been found be the most competitive reaction on noble metal base (Rh, Ru, Ir, Pt, Pd) catalysts at “low temperature” (T<750° C.) and high O₂ partial pressure. These are typically the conditions produced at the beginning of the catalytic beds in tubular reactors operated at “high” contact time values (above 1 s). In these cases, we found that the thermal profiles of the reaction environments are determined by the strongly exothermic reaction [12] with a minor contribution of reactions [14] and [14], followed by the strongly endothermic steam-CO₂ reforming reactions [1] and [10]. These conditions originate very large axial temperature gradients and moreover, the energy release associated to total combustion also determines the propagation of the heterogeneous reactions into the gaseous phase originating a rather unselective radical chemistry leading to unsaturated molecules and soot formation.

Indeed, we have found that with tubular reactors the catalytic partial oxidation reactions cannot be performed at high pressures since reaction [12] cannot be controlled and propagate reactions into the gaseous phase with the risk of flame ignition and producing radical reactions leading to the formation of unsaturated hydrocarbons and soot.

Instead, it has been found that the solid catalyst temperatures can reach values higher than 1000° C. while the gas remains relatively cool by utilizing a reaction environment geometry that allows to reduce the contact time at the entrance of the catalytic bed to few milliseconds and that allows the expansion of the reaction volume when the temperature and the mole flow increases due to the progressing of the reaction. This effect is obtained by adopting a truncated shape geometry of the catalytic bed and catalyst geometrical features that allow to reduce the pressure drop inside the reaction zone.

Accordingly, it has been found that in these short contact time conditions, the methane conversion largely depends on the O₂/C ratios while is almost unaffected by the addition of steam and CO₂. This addition instead modifies the H₂/CO ratios in the produced synthesis gas clearly indicating that the reactivity is largely determined by the direct partial oxidation [13] and by the RWGS reaction [14].

FIG. 1 shows qualitative pictures of the Enthalpy/Temperature profiles obtained at (A) low mass velocity, high contact time, tubular geometry of the catalytic bed and (B) high mass velocity, short contact time, truncated cone geometry of the catalytic bed.

FIG. 2 shows the main zones of the SCT-CPO reactor including a truncated cone reaction zone.

The cylindrical first portion includes a mixing inlet zone and a first thermal shield pre-heating zone.

The central second portion includes a reaction zone,

The cylindrical third portion includes a second thermal shield and a reactor exit zone.

The angle α shown in FIG. 9 is clearly lower than 85° and preferentially comprised between 75° and 30°.

The other geometrical features namely the: i) truncated cone inlet radius R1, ii) the truncated cone exit radius R2, iii) the truncated cone length L and the catalytic bed filling are designed for allowing pressure drop values (ΔP) inside the catalytic bed between 0.1 and 10 ATM and preferentially between 0.5 and 5 ATM.

To this purpose the ratios R1/R2 have to be comprised between 0.9 and 0.1 and preferentially between 0.8 and 0.4 and the shapes of the filling of the catalyst bed need to be defined for minimizing the pressure drop conditions by utilizing pelletized or monolith structures and their combinations.

It has also been noted that the existence of a non-thermal equilibrium between the gas and the solid phases and this has been explained considering that the chemical heat generated at the surfaces and emitted by radiation, is absorbed, and scattered much better by the solid than by the gaseous phase and is transferred along the catalytic bed from the hotter towards the cooler points smoothing the solid surface temperatures.

The main experimental observations on the thermo-chemical properties of the SCT environments, performed after optimization of the reaction environment characteristics, are synthesized as follows:
i) the temperature of the solid phase raises steeply at the beginning of the bed and the temperature profiles are smoothed through radiative and conduction mechanisms in the axial and radial directions;
ii) temperature differences are originated between the gas and the solid phases;
iii) part of the surface temperatures results higher than the adiabatic temperatures;
iv) gas temperatures are always lower than the adiabatic temperatures and gradually increase from the entrance to the exit of the bed

It has also been found that part of the reaction heat is transferred towards the incoming reactants inside the first thermal shield zone and in this way a reactant pre-heating internal to the reactor is also achieved.

In the following we describe process schemes based on the possibility of utilizing such a SCT-CPO reactor for treating a gaseous hydrocarbon feedstock containing relevant amounts of CO₂ (between 1-50% v/v and preferentially between 5-35% v/v) also at high pressure (between 1-60 ATM and preferentially between 5-35 ATM) without incurring in the carbon formation reactions. Moreover, since SCT-CPO requires a moderate pre-heating (between 50-450° C. and preferentially between 15° and 400° C.), this can be provided with gas-gas heat exchangers recovering the heat from the produced synthesis gas.

More in detail it is specified that the gaseous hydrocarbon stream could be constituted by methane, natural gas, acid gas, natural gas liquids, well head gas, refinery and chemical plants off gases including pyrolysis gases, biogas and combinations thereof.

Accordingly, in the described process solutions, the CO₂ emissions are mainly related to the E.E. consumption required for:
i) compressing the feedstock (compression energy that would be required for any syngas production technology),
ii) for obtaining the 02 flow with an Air Separation Unit (ASU),
iii) in some cases, for performing steam and CO₂ electrolysis and in some others for operating an electrified reverse water gas shift reactor (E-RWGS)

Noteworthy, when steam electrolysis is included in the process architectures here described, not only the H₂ stream participate to the process flows but also the co-produced O₂ streams are utilized by the SCT-CPO reactor, and this increases the energy efficiency of the overall solutions.

The scheme described in FIG. 3 describes a solution for producing acetic acid from a CO₂ rich hydrocarbon stream by producing, via SCT-CPO, a synthesis gas with a low H₂ vs CO ratio (ca. 1 v/v) and by treating the obtained synthesis gas with a CO removal unit for producing a stream with a composition suitable for the methanol synthesis and hence with a methanol module M=(H₂—CO₂)/(CO+CO₂) v/v ca. 2 and a CO stream. This last is utilized for methanol carbonylation for producing Acetic Acid with commercial technologies such those mentioned in Table 1.

TABLE 1

| | | | MeOH | |
|---|---|---|---|---|
| TECHNOLOGY | T | P | cons | CO cons |
| (Process Economic Program) | (C.) | (ATM) | (kg/kg) | (Nm³/Ton) |
| MONSANTO (Rh BASED | 200 | 69 | 0.545 | 424 |
| CATALYST) | | | | |
| BP - CATIVA (Ir BASED | 195 | 28 | 0.539 | 376 |
| CATALYST) | | | | |
| CHIYODA - CT ACETICA | 195 | 32 | 0.556 | 386 |
| (Rh BASED CATALYST) | | | | |
| CELANESE AO PLUS (Rh | 200 | 30 | 0.523 | 376 |
| BASED CATALYST) | | | | |

In this scheme, as mentioned above, the CO₂ emissions are mainly related to the E.E. consumption required for compressing the feedstock (compression energy that would be required for any syngas production technology) and for obtaining the O₂ flow.

If it is assumed, for the purpose of this discussion, that:
i) the simplified reactivity features are described by eq. [15] by utilizing a CH₄+CO₂ mixture in which the CO₂ content corresponds to 30% v/v, and
ii) the O₂ flow would be produced with an Air Separation Unit (ASU) in a scenario in which carbon intensity for E.E. production will vary between 50 gCO₂/kWh-200 gCO₂/kWh (current EU emissions range).

The emissions would vary between 0.03-0.12 mole of emitted CO₂ per mole of consumed CO₂ and would allow a strong reduction with respect to the CO₂ emissions associated to the heating furnaces of the dry reforming units (ca. 0.6 moles of emitted CO₂ per mole of consumed CO₂ by considering reactions [1]] and [12]).

$${CH}_{4} + 0.56 {O}_{2} + 0.43 {CO}_{2} = 1.45 {H}_{2} + 1.43 CO + 0.55 {H}_{2} O \\ ΔH ° = - 47.04 kJ / mole \qquad [ 15 ]$$

The scheme of FIG. 4 describes instead solutions for integrating CO₂ rich streams into acetic acid molecules. The scheme includes the production, via SCT-CPO, of a syngas with low H₂ vs CO ratios (H₂/CO ca 1 v/v) with two SCT-CPO reactors (A) and (B) operating in parallel.

The components of the syngas produced by reactor (B) are separated and the H₂ containing stream is added to the syngas produced by reactor (A) for achieving a composition suitable for the methanol synthesis, while the CO is used for the following methanol carbonylation and the achievement of acetic acid molecules.

The scheme of FIG. 5 describes solutions for integrating CO₂ rich streams into acetic acid molecules by adjusting the synthesis gas composition produced with SCT-CPO by adding an H₂ flow produced by steam electrolysis obtained with Alkaline Electrolysis (AE), Polymer Membrane Electrolysis (PEME) but also with Solid Oxide Electrolysis Cells (SOEC).

In this case the associated O₂ production is utilized in the SCT-CPO reactor combined, if it is the case, with the O₂ produced by an ASU. Clearly the contribution of the H₂ and O₂ addition obtained with electrolysis need to be evaluated as function of the associated electric energy consumption and of the amount of CO₂ included in the SCT-CPO feedstock.

The scheme also considers the possibility of producing a CO rich stream from CO₂ and H₂ with an electrified reverse water gas shift reactor (E-RWGS) with solutions such those described in S. De Sarkar, K. Aasberg-Petersen, T.S. Christensen, P.M. Mortensen, “Conversion of CO₂ and H₂ to synfuels”; WO 2022/079098 A1. Indeed, quite recently it has been estimated that the electrification of the RWGS reactor would allow the production of CO rich mixtures avoiding the CO₂ emissions of the same reactors utilizing heating furnaces thus allowing the definition of CO₂ sink processes.

As in FIGS. 3 and 4 also in the scheme of FIG. 5, the MeOH and the CO streams are then combined for producing Acetic Acid with methanol carbonylation technologies.

The scheme described in FIG. 6 is also based on the possibility of SCT-CPO of treating a gaseous hydrocarbon feedstock with relevant amounts of CO₂ also at high pressure (e.g., 30 bars) without incurring in the soot formation reactions an on the moderate pre-heating of the reactants avoiding the fire heaters CO₂ emissions. As in the process scheme of FIG. 5 the composition of the syngas is adjusted for achieving the methanol module M ca. 2 v/v by adding an H₂ flow produced by steam electrolysis obtained with Alkaline Electrolysis (AE), Polymer Membrane Electrolysis (PEME) but also with Solid Oxide Electrolysis Cells (SOEC). However, differently from the scheme of FIG. 5 the scheme of FIG. 6 considers the possibility of producing a CO rich stream with an SOEC fed with a CO₂ stream. As in FIGS. 3-5 the MeOH and the CO streams are then combined for producing Acetic Acid with methanol carbonylation technologies.

The schemes of FIGS. 7-10 include the same solutions described in FIGS. 3-6 but in this case the methanol and the CO streams are produced for feeding methanol oxidative carbonylation reactions for obtaining DMC.

The schemes of FIG. 11 include the same solutions of FIG. 3 for producing a syngas with a H₂ vs CO ratio of 1.0-1.5 v/v integrated in an hydroformylation process (A) for oxo-aldehyde productions and (B) for oxo-alcohols productions.

1-17. (canceled)

18. A method of producing one or more of alcohols, carboxylic acids, carbonyl compounds, and carbonates, the method comprising:
a) producing a syngas via a short contact time catalytic partial oxidation process which uses a feedstock comprising an organic component and an inorganic component, wherein:
i) the organic component is a mixture comprising carbon dioxide included in one or more of natural gas, off-gases of industrial chemical or refining activities, and blast furnace gases, coke oven gases, direct reduction gases produced in the reduction of iron ores activities, biogas and carbon dioxide captured from gaseous streams, the carbon dioxide being from 1 to 50% v/v of the mixture;
ii) the inorganic component comprises one or more of air, oxygen, and air enriched with oxygen, the oxygen being obtained from electrolysis of water or from air;
iii) the short contact time catalytic partial oxidation process is carried out in a reactor comprising a first portion having a cylindrical shape including a mixing inlet zone and a first thermal preheating shield zone, a second portion having a truncated cone shape containing a catalytic bed, and a third part having a cylindrical shape with a diameter greater than the diameter of the first cylindrical portion, including a second thermal shield zone and a reactor exit zone, wherein:
a. the first thermal shield zone is pre-heated at a temperature from 50 to 450° C.; and
b. the short contact time catalytic partial oxidation is carried out on the catalytic bed at a gas hourly space velocity from 15,000 to 500,000 h⁻¹ and at a pressure from 1 to 60 atm;
iv) whereby the syngas obtained from the reactor has a hydrogen/CO ratio from 0.5 to 2;
b) using the syngas for the production of a compound selected from the group consisting of carboxylic acids, alcohols, carbonyl compounds, and carbonates.

19. The method of claim 18, wherein the carbon dioxide in step a)i) is from 5 to 35% v/v of the mixture.

20. The method of claim 18, wherein the short contact time catalytic partial oxidation is carried out on the catalytic bed at a pressure from 5 to 35 atm.

21. The method of claim 18, wherein the alcohol of step b) is methanol.

22. The method of claim 21, wherein the methanol is fed to a reactor for the carbonylation of methanol to produce carboxylic acids and carbonyl compounds.

23. The method of claim 18, wherein the synthesis gas is fed to an olefins hydroformylation reactor for producing aldehyde compounds.

24. The method of claim 23, wherein the aldehyde compounds are fed to a hydrogenation reactor for producing corresponding alcohols.

25. The method of claim 18, wherein the syngas has a hydrogen/CO ratio from 0.7 to 1.5.

26. The method of claim 25, wherein the syngas has a hydrogen/CO ratio from 0.8 to 1.3.

27. The method of claim 18, wherein the oxygen from air of the step a)ii) is obtained via a process selected from cryogenic air separation, vacuum pressure swing adsorption, and membrane separation.

28. The method of claim 18, wherein the oxygen of the step a)ii) is obtained from both air and from electrolysis of water.

29. The method of claim 18, wherein the syngas produced in the step a) is treated in a pressure swing adsorption unit to separate a portion of the CO, and the separated portion of CO is fed to the step b) to carry out a carbonylation reaction.

30. The method of claim 18, wherein the feedstock is pre-heated in the step a)iii) via a process selected from:
recovering heat from the produced syngas with gas-gas heat exchangers; and
transferring to the feedstock inside the reactor part of the reaction heat before the feedstock reaches the reaction zone.

31. The method of claim 18, wherein the syngas is produced in two separate short contact time catalytic partial oxidation reactors operating in parallel, with the syngas produced in one of the two reactors which is treated to separate hydrogen and CO, and with the hydrogen which is mixed to the syngas produced in the other short contact time catalytic partial oxidation reactor and fed to the step b) for the synthesis of methanol, and the CO is fed to the step c) to produce carboxylic acids and carbonyl compounds.

32. The method of claim 18, wherein the syngas is produced also in a reverse water gas shift reactor which uses a feedstock comprising a mixture of hydrogen and carbon dioxide, and wherein the CO contained in the syngas produced in the reverse water gas shift reactor is used to produce carboxylic acids and carbonyl compounds.

33. The method of claim 32, comprising a Solid Oxide Electrolysis Cells (SOEC) fed with a stream containing carbon dioxide and producing a stream containing CO which is used to produce the carboxylic acids and carbonyl compounds.

34. The method of claim 18, wherein the carboxylic acid is acetic acid.

35. The method of claim 18, wherein the carbonyl compound is dimethyl carbonate.

36. An apparatus for producing carboxylic acids and carbonyl compounds according to the method of claim 18, comprising:
a short contact time catalytic partial oxidation reactor comprising a first part having a cylindrical shape including an inlet, a mixing zone and a first thermal shield zone, a second part having a truncated cone shape containing a catalytic bed, and a third part having a cylindrical shape with a diameter greater than the diameter of the first cylindrical portion, including a second thermal shield zone, wherein:
in the second part having the shape of a truncated cone the upper base is smaller than the bottom base;
the upper base of the truncated cone is joined to the first cylindrical portion and the bottom base is joined to the third cylindrical part; and
the external angle (α) of the truncated cone at the upper base is lower than 85°.

37. The apparatus of claim 36, wherein the upper base having the shape of a truncated cone has a diameter R1 and the bottom base has a diameter R2, wherein the ratio R1/R2 is from 0.9 to 0.1.
